(19) United States
(12) Patent Application Publication
(10) Pub. No.: US 2008/0306337 A1
(43) Pub. Date: Dec. 11, 2008

(54) Characterization of a Near-Infrared Laparoscopic Hyperspectral Imaging System for Minimally Invasive Surgery

(75) Inventors: Edward H. Livingston, Dallas, TX (US); Karel Zuzak, Arlington, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/137,220

(22) Filed: Jun. 11, 2008

Related U.S. Application Data
(60) Provisional application No. 60/943,260, filed on Jun. 11, 2007.

(51) Int. Cl. A61B 1/04; G06K 9/46

(52) U.S. Cl. 600/109; 382/191

(57) ABSTRACT

The present invention includes an apparatus and methods for use of a hyperspectral surgical laparoscope that includes an illuminated laparoscope; a liquid crystal tunable filter generally center-mounted on the laparoscope and positioned to collect back-reflected light from a target; a relay lens generally center-mounted on the laparoscope to focus light from the liquid crystal tunable filter; and a focal plane array generally center-mounted on the laparoscope, wherein light that is reflected from the target is imaged on the focal plane array and captured as a digital data cube.

### CROSS-REFERENCE TO RELATED APPLICATIONS

This application claims priority to U.S. Provisional Patent Application No. 60/943,260, filed Jun. 11, 2007, the entire contents of which are incorporated herein by reference.

### TECHNICAL FIELD OF THE INVENTION

The present invention relates in general to the field of surgery and imaging and, more particularly, to an apparatus, method and system for near-infrared laparoscopic hyperspectral imaging system for minimally invasive surgery.

### STATEMENT OF FEDERALLY FUNDED RESEARCH

None.

### BACKGROUND OF THE INVENTION

Without limiting the scope of the invention, its background is described in connection with hyperspectral imaging.

A number of hyperspectral imaging systems have been developed that are in current use. One such system is taught in U.S. Pat. No. 7,149,366, issued to Sun for a high-definition hyperspectral imaging system. Briefly, a compact high-definition hyperspectral imaging system (HDHIS) is taught for light aircraft remote sensing to perform concurrent pushbroom hyperspectral imaging and high-resolution photographic imaging. The HDHIS includes a sensor head having a hyperspectral scanner and a CCD digital camera. An airborne computer interfaces with the sensor head to provide data acquisition including hyperspectral quick view images and control functions.

Yet another hyperspectral system in taught in U.S. Pat. No. 6,992,775, issued to Soliz, et al., for a hyperspectral retinal imager. Briefly, an ophthalmic instrument (for obtaining high resolution, wide field of area hyperspectral retinal images for various sized eyes) includes a fundus retinal imager, (which includes optics for illuminating and imaging the retina of the eye); an apparatus for generating a real time image of the area being imaged (and the location of the hyperspectral region of interest); a high efficiency spatially modulated common path Fourier transform hyperspectral imager, a high resolution detector coupled optically to the hyperspectral and fundus imager optics; and a computer (which is connected to the real time scene imager, the illumination source, and the high resolution camera) including an algorithm for recovery and calibration of the hyperspectral images.

Another such system is taught in U.S. Pat. No. 6,937,885, issued to Lewis, et al., which teaches a multispectral/hyperspectral medical instrument. Briefly, a medical instrument includes a first-stage optic responsive to a tissue surface of a patient; a spectral separator optically responsive to the first stage optic and having a control input; an imaging sensor optically responsive to the spectral separator and having an image data output; and a diagnostic processor having an image acquisition interface with an input responsive to the imaging sensor and a filter control interface having a control output provided to the control input of the spectral separator.

U.S. Pat. No. 6,640,132, issued to Freeman, et al., teaches a forensic hyperspectral apparatus and method. The invention relates to a portable imaging device, such as hyperspectral imaging devices, useful for forensic and other analysis, and methods for using these devices. Devices of the invention provide images and patterned data arrays representing images in multiple discrete spectra that can then be summed or processed to allow for detection of patterns or anomalies in the data collected.

Finally, U.S. Pat. No. 6,160,618 issued to Gamer, teaches a hyperspectral slide reader. Briefly, an apparatus and method for analyzing samples on a slide is taught that includes a slide mover positioned to hold a slide, a imaging spectrometer positioned in the path of light from the slide to split the light line into a light array, a light amplifier may be positioned between the imaging spectrometer and a camera, is disclosed. The camera can detect the entire spectrum of light produced by the imaging spectrometer.

### SUMMARY OF THE INVENTION

The present inventors developed and characterized a new imaging platform for minimally invasive surgical venues, specifically, a system to help guide laparoscopic surgeons to visualize the macro and micro anatomy of a tissue, e.g., biliary anatomy. The platform is a novel combination of a near-infrared hyperspectral imaging system coupled with a conventional surgical laparoscope. Intra-operative tissues are illuminated by optical fibers arranged in a ring around a center-mounted relay lens collecting back-reflected light from tissues to the hyperspectral imaging system. The system includes a focal plane array (FPA) and a liquid crystal tunable filter (LCTF), which is continuously tunable in the near-infrared spectral range of 650-1100 nm with the capability of passing light with a mean bandwidth of 6.95 nm and the FPA is a high-sensitivity back-illuminated, deep depleted charged coupled device.

For example, placing a standard resolution target 5.1 cm from the distal end of the laparoscope, a typical intraoperative working distance, produced a 7.6 cm diameter field of view with an optimal spatial resolution of 0.24 mm. In addition the system's spatial and spectral resolution and its wavelength tuning accuracy are characterized. The spectroscopic images are formatted into a three dimensional hyperspectral image cube and processed using principle component analysis. The processed images enhance the contrast between chemically different anatomical structures and help identify the main molecular chromophores inherent to each tissue. The principal component images were found to enhance the contrast of the swine gallbladder and biliary structures from surrounding tissues, in real time, during cholecystectomy surgery. Furthermore, it is shown that surgeons can interrogate selected image sub-regions for their molecular composition during surgery and before any invasive action is undertaken.

More particularly, the present invention includes a hyperspectral surgical laparoscope having an illuminated laparoscope; a liquid crystal tunable filter generally center-mounted on the laparoscope and positioned to collect back-reflected light from a target; a relay lens generally center-mounted on the laparoscope to focus light from the liquid crystal tunable filter; and a focal plane array generally center-mounted on the laparoscope, wherein light that is reflected from the target is imaged on the focal plane array and captured as a digital data cube.

In one aspect, the illuminated laparoscope delivers continuously tunable light in the near-infrared spectral range of 650-1100 nm. One example of a light is in the focal plane array and has a mean bandwidth of 695 nm. The focal plane array may be a high-sensitivity, back-illuminated, deep depleted charged coupled device. The digital data cube may be formatted into a three dimensional hyperspectral image cube and processed using principle component analysis. The digital data cube may also be processed to enhance the contrast between chemically different anatomical structures due to chromophores inherent to the target, chromophores that have been added to the target or both. Examples of targets include an intraperitoneal tissue, a gall bladder or a bile duct. The laparoscope may be connected to a light source comprising a visible to near infrared liquid light guide, e.g., a 250-W quartz-tungsten-halogen broadband source. The apparatus may also include an ultraviolet radiation filter positioned between a light source and the target. The liquid crystal tunable filter may be an electronically controlled and continuously tunable filter with 150 ms tuning response time and a clear 20 mm aperture.

Another embodiment of the present is a near-infrared hyperspectral surgical laparoscope that includes an near-infrared illuminated laparoscope; a liquid crystal tunable filter generally center-mounted on the laparoscope and positioned to collect back-reflected light from a target; a relay lens generally center-mounted on the laparoscope to focus light from the liquid crystal tunable filter; and a focal plane array comprising a high-sensitivity, back-illuminated, deep depleted charged coupled device generally center-mounted on the laparoscope, wherein light that is reflected from the target is imaged on the focal plane array and captured as a digital data cube. In one aspect, the illuminated laparoscope delivers continuously tunable light in the near-infrared spectral range of 650-1100 nm. One example of a light is in the focal plane array and has a mean bandwidth of 695 nm. The focal plane array may be a high-sensitivity, back-illuminated, deep depleted charged coupled device. The digital data cube may be formatted into a three dimensional hyperspectral image cube and processed using principle component analysis. The digital data cube may also be processed to enhance the contrast between chemically different anatomical structures due to chromophores inherent to the target, chromophores that have been added to the target or both. Examples of targets include an intraperitoneal tissue, a gall bladder or a bile duct. The laparoscope may be connected to a light source comprising a visible to near infrared liquid light guide, e.g., a 250-W quartz-tungsten-halogen broadband source. The apparatus may also include an ultraviolet radiation filter positioned between a light source and the target. The liquid crystal tunable filter may be an electronically controlled and continuously tunable filter with 150 ms tuning response time and a clear 20 mm aperture.

Yet another embodiment is a method of non-invasive imaging method to identify a biliary tree structure that includes imaging the biliary tree structure with a laparoscope using a liquid crystal tunable filter (LCTF) positioned to collect back-reflected light from a target; a relay lens center-mounted on the laparoscope to focus light from the liquid crystal tunable filter; and a focal plane array (FPA), wherein light that is reflected from the target is imaged on the focal plane array and captured as a digital data cube; and processing the digital data cube to enhance the contrast between chemically different anatomical structures due to chromophores at the target. The digital data cube may be processed to enhance the contrast between chemically different anatomical structures due to chromophores inherent to the target or to enhance the contrast between chemically different anatomical structures due to chromophores that have been added to the target. In one aspect, the laparoscope is connected to a light source comprising a visible to near infrared liquid light guide. One example of a light source is a 250-W quartz-tungsten-halogen broadband source.

The present invention also includes a method of conducting a cholecystectomy and a system for laparoscopic cholecystectomy by imaging laparoscopic surgery imaging the intraportal structure with a near infrared laparoscope that includes a liquid crystal tunable filter (LCTF) positioned to collect back-reflected light from a target; a relay lens center-mounted on the laparoscope to focus light from the liquid crystal tunable filter; and a focal plane array (FPA), wherein light that is reflected from the target is imaged on the focal plane array and captured as a digital data cube; processing the digital data cube to enhance the contrast between chemically different anatomical structures due to chromophores at the target; and removing those tissue that are identified in need of removal.

### BRIEF DESCRIPTION OF THE DRAWINGS

For a more complete understanding of the features and advantages of the present invention, reference is now made to the detailed description of the invention along with the accompanying figures and in which:

FIG. 1 shows an in-vivo near-infrared laparoscopic hyperspectral imaging system.

FIGS. 2A to 2C are graphs that show the near-infrared LCTF (NIR-50970a) calibration (2A), the diamonds indicate the experimental values and the dashed line indicating the regressed line. The x axis represents the center wavelengths, CW, the LCTF was expected to transmit, and the y axis depicts actual measured center wavelengths transmitted. FIG. 2B is the near-infrared LCTF (NIR-50970a) bandwidth characteristics measured at the FWHM shown at every 20 nm as the filter is tuned through its wavelength range. FIG. 2C shows the spatial resolving power of the laparoscopic hyperspectral imaging system determined by a contrast transfer function analysis. The percent contrast, C, is plotted as a function of spatial resolution (mm). Employing the full FPA chip (1340×400) binning 1×1 (diamonds); (640×200) binning 2×2 (squares); (447×133) binning 3×3 (triangles); (355×100) 4×4, (x's). Placing a 1951 USAF resolution target 5.1 cm from the laparoscope with the LCTF tuned to a center passband of 750 nm and utilizing the full FPA chip binning pixels 1×1 provides a 0.24-mm spatial resolution based upon the Rayleigh criterion, represented by the dotted line.

FIG. 3 is a laparoscopic picture using a conventional digital camera and endoscope (Karl Storz) during closed laparoscopic procedures picturing the gallbladder (3A) and liver (3B).

FIG. 4 is a second principle component spectrum (4A) and corresponding principle component image (4B) of the gallbladder and liver collected with the laparoscopic hyperspectral imager during closed cholecystectomy. Fifth principle component spectrum (4C) and corresponding principle component image (4D) of the gallbladder and liver collected with the laparoscopic hyperspectral imager during closed cholecystectomy.

FIGS. 5A to 5C show that the fifth principle component image (5A) was found to provide the best discriminating capacity for contrasting gallbladder from liver, which was confirmed by the morphology and spectra. Measured spectra within the area indicated by each sampling box in (5A) were average and plotted. Spectrum (5B) contains an absorption peak at 760 nm (1) characteristic of deoxyhemoglobin, followed a broad banded absorption beyond 800 nm (2) typical of oxyhemoglobin, with lipids at 930 nm (3) broadening the main 970 nm water peak (4) a molecular mixture consistent with known constituents contained within gallbladder. In contrast, spectrum (5C) from the liver measured absorbance from deoxyhemoglobin (1) and oxyhemoglobin (2).

FIG. 6 shows spectra (A1-A3 and B1-B3) sampled from gallbladder (column A) and liver (column B) from three different pigs (rows 1-3). The salient spectral features for oxyhemoglobin, deoxyhemoglobin, lipids and water in each of the reflectance spectra can be identified by the corresponding peaks in FIGS. 5B and 5C.

FIG. 7 shows a principle component analysis (PCA) image visualizing the Gallbladder, liver, and cystic duct and their associated spectra.

FIG. 8 shows fat and its associated spectrum lying along the stomach within a PCA image.

FIG. 9 shows a PCA image from data collected with the infrared hyperspectral imager is compared to a conventional black and white photo of the gallbladder taken with a standard surgical Karl Storz laparoscopic system.

FIG. 10 shows the laparoscopic hyperspectral data and its principle component image visualizing intraportal structures (artery, vein and bile duct) through connective tissue without using contrast agents.

FIG. 11 shows that a principle component image, left, has greater specificity for imaging the human bile duct through undissected connective tissue of the porta than a standard black and white picture, right image.

FIG. 12 shows individual measured spectra sampled from the human gallbladder, biliary tree and fat indicate the system is capable of differentiating fat and bile.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. NIR-Near Infrared, LCTF-Liquid Crystal Tunable Filter, QTH-Quartz Tungsten Halogen, FPA-Focal Plane Array, FWHM-Full Width and Half Maximum, PCA-Principle Component Analysis, CCD-Charge Coupled Device.

Disease of the gallbladder often requires its surgical removal, a procedure called cholecystectomy. Cholecystectomy is one of the most commonly performed operations in the United States with more than 400,000 cases being performed annually.^{1,2} The preferred method is a minimally invasive procedure known as closed laparoscopic cholecystectomy, which requires two to three small incisions, approximately 10 mm in diameter, in the abdomen to allow the insertion of surgical instruments and a small camera. The camera provides the surgeon with images from inside the body and reduces a 200 mm incision to 10 mm. Traditional video imaging of tissues through an endoscope usually renders poor image contrast. Currently for identification of the biliary tree during difficult procedures the only imaging modality is an intra-operative cholangiography first devised by Mirizzi in 1937.³ It is a time consuming and surgically expensive technique involving the dissection of the cystic duct, incising it, threading a catheter, injecting radioactive contrast material and obtaining radiographs during surgery.

There is a pressing need for a non-invasive imaging method that can identify biliary tree structures during the operation in a user friendly and expedient manner, thus making surgery safer and reducing operating time. We sought to meet this need by developing an endoscope-based hyperspectral imaging system enabling a surgeon to visualize tissues with enhanced contrast on a video monitor. In addition, the real-time, in-vivo hyperspectral imaging methodology enables the non-invasive interrogation of tissues for their chemical composition.

The hyperspectral imaging method measures spectrally-resolved light intensity for each image pixel simultaneously. Multiple images are obtained at discrete, typically sequential, narrow wavelength bands over a spectral range of interest. The resulting data set is formatted as a three-dimensional (3-D) data matrix, where spatial information is collected in the x-y plane and wavelength data on the z plane.⁴ The measured light intensity in each wavelength band is determined by the tissue optical properties, the illumination geometry and the overall detection sensitivity of the system. The optical properties of each tissue type are described by its scattering and absorption coefficients, both of which are functions of wavelength.⁵ The spectral dependence of optical properties results in differences in the mean tissue depths being visited by different wavelength photons, with near-infrared photons penetrating the deepest.⁶ Therefore, the detected hyperspectral images are a convolution of the absorbance spectra of resident chromophores from within the tissues. We have previously demonstrated that spectra measured, in-vivo, from within the skin over the visible wavelength range can be deconvolved and gray scale encoded for the percentage of oxyhemoglobin (HbO₂) and deoxyhemoglobin (Hb).^{4, 7, 8, 9} Here, we extend our previous work with the construction and characterization of a hyperspectral imaging system capable of detecting photons in the near-infrared (NIR) wavelength range. In addition to traveling deeper in tissues, near-infrared light is known to sample significant absorbance regions in the spectra Hb, HbO₂, water and lipid,¹⁰ inherent tissue chromophores, that can be used for differentiating anatomical structures based on their chemical composition. A proof of principle study demonstrating improved visualization of the biliary system during laparoscopic cholecystectomy in three 60 pound swine is presented.

Instrument Design and Construction. Recent developments in focal-plane-array detectors for spectroscopic imaging along with the implementation of liquid crystal tunable filters have enabled constructing a novel device for laparoscopic hyperspectral imaging in humans.¹¹⁻¹⁹ The construction of such a device and its operating characteristics are disclosed.

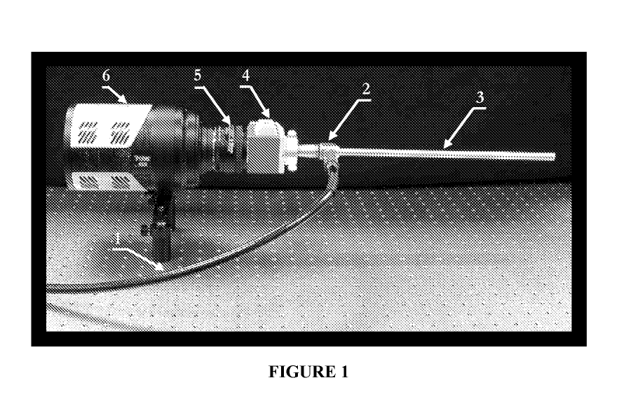

FIG. 1 displays the configuration of the laparoscopic hyperspectral imager consisting of a source, a tunable filter, a detector and a variety of optics. Broad band light from a quartz-tungsten-halogen (QTH) light source is transferred by a liquid light guide (LLG) 1 into the abdominal cavity via laparoscope fiber optics 2. The diffuse reflectance is guided by the center mounted laparoscope relay optics 3 back through the LCTF 4 and a lens 5, which focuses the tissue image onto the FPA detector 6.

In operation, broadband light can delivered into the operative field via a conventional surgical laparoscope (Karl Storz, Germany) connected to the source by a visible to near infrared (NIR) liquid light guide (LLG)(Oriel, Stratford, Conn.). The LLG's large-diameter core (8 mm) allows for a high light throughput ranging from visible through the near infrared (NIR) spectrum (>80% transmittance 450-1600 nm) while filtering ultraviolet radiation, and reducing the potential for light-induced tissue damage. A radiometric power supply (Oriel, Stratford, Conn.) provides a constant current to the source maintaining a stable lamp output by minimizing light ripple (<0.05% rms) and spectral noise. The light source, a 250-W quartz-tungsten-halogen (QTH) broadband source, was mounted in a versatile protective enclosure (Oriel, Stratford, Conn.) with a rear reflector maximizing light for tissue illumination. A series condenser and fiber bundle focusing assembly (Oriel, Stratford, Conn.) is used to efficiently focus light radiating from the source onto the LLG.

The viewing optics of the laparoscope directs light reflecting from tissue to the liquid crystal tunable filter (LCTF, Cambridge Research & Instrumentation, Boston, Mass.). The LCTF is an electronically controlled and continuously tunable filter with 150 ms tuning response time and a clear 20 mm aperture for producing spectroscopic images rapidly. A 50 mm, f/1.4 lens (Nikon, Tokyo, Japan) focuses each spectroscopic image onto a digital focal plane array, FPA. The PIXIS 400 BR FPA (Princeton Instruments, Trenton, N.J.) is a fully integrated system with permanent vacuum and thermoelectric cooling down to −75° C. producing a minimal dark current of 0.25 e⁻/p/s, using a high performance back-illuminated charge-coupled device, CCD. The system incorporates deep-depletion technology to improve quantum efficiency and employs interference filters to eliminate etaloning at NIR wavelengths. The FPA is formatted into a 1340×400 pixel matrix of 20×20 μm pixel detectors when coupled with the LCTF is capable of registering 536,000 simultaneously acquired spectra. The FPA has a spectral response ranging from UV to the NIR (200-1100 nm) with a 16 bit analog-digital converter, ADC, digitizing at a maximum rate of 2 MHz. LCTF tuning, image acquisition, and data storage are managed by a computer program written in the laboratory and compiled by V⁺⁺ (Princeton Instruments, Trenton, N.J.). A high-end laptop computer (Dell Latitude D610; Austin, Tex.) manages the instrument control, spectral image acquisition and processing. Image visualization during surgery was performed using routines written in the laboratory using the Matlab software environment (Ver. R2006b, Mathworks, Natick, Mass.).

System Characterization: Tuning Accuracy of the LCTF. The operating characteristics of the Laparoscopic Hyperspectral Imaging System were evaluated for in-vivo surgical utility. The spectral tuning accuracy for the LCTF (serial number NIR50970a) was determined using a calibrated PerkinElmer Spectrometer (PerkinElmer, Wellesley, Mass.). The LCTF was placed in the collimated optical path of the spectrometer, tuned to a specific center wavelength and the passband of transmitted light intensity was measured. This procedure was repeated for a series of sequential wavelengths spanning the near-infrared range of the LCTF from 650 nm to 1050 nm at 50 nm increments. Wavelength accuracy is taken to be the actual center wavelength, CW_{actual}, to be correct within the nominal, CWₙₘₗ.

$$C {W}_{actual} = C {W}_{nm l} \pm ( \frac{{Bandwidth}_{nm l}}{8} + 0.5 nm ) \qquad ( 1 )$$

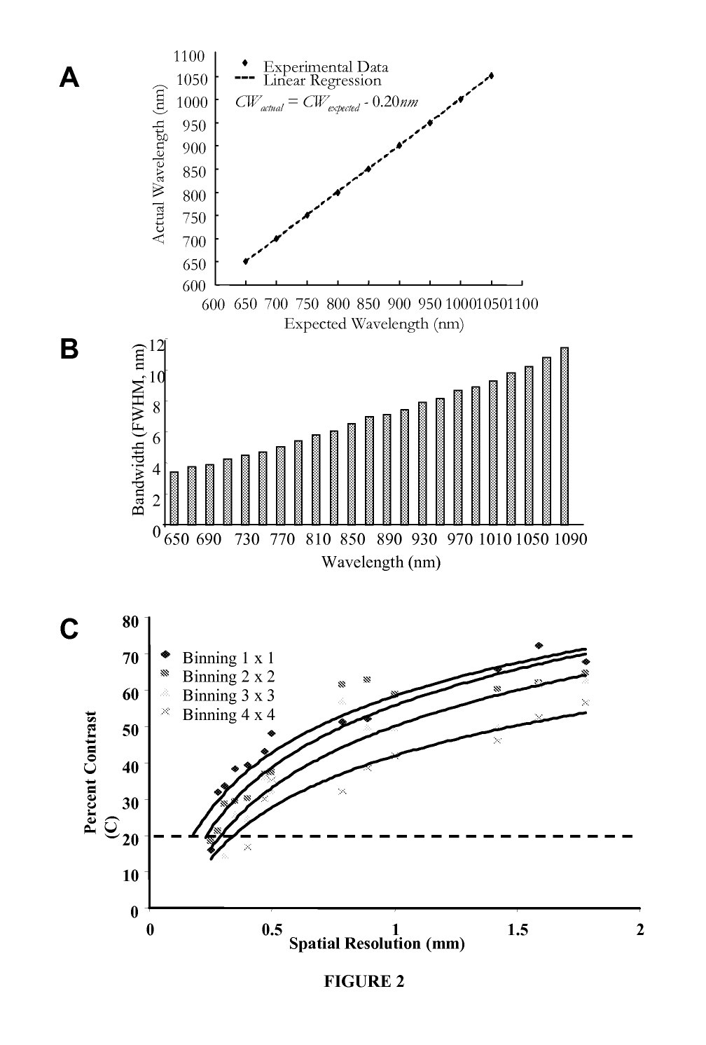

On average this LCTF was found to tune 0.17 nm above the expected wavelength, which is well within the manufacturer's specification of ±1.38 nm. The expected wavelengths sent electronically to the LCTF controller were plotted against the experimentally measured center wavelengths transmitted by the LCTF, FIG. 2A. Linearly regressing the experimentally measured center wavelengths, CW_{actual}, against the expected, CW_{expected}, the calibration equation,

CW_{actual}=CW_{expected}−0.20 nm (2)

having an R²=0.99, and Pᵥₐₗ<0.05. Incorporating the calibration equation into the V⁺⁺ computer program managing the data collection increased the accuracy of the LCTF tuning to the desired center wavelength.

LCTF Bandwidth Characteristics. The near-infrared LCTF bandwidth characteristics were measured at the full width at half-maximum (FWHM) determined as the spectral separation between the two points where the filter's transmission attains 50% of the peak value. The FWHM varies linearly as the function of wavelength ranges from 3.4 nm at 650 nm to 11.4 nm at 1090 nm, with an average of 6.95 nm across the range (650-1100 nm) of the LCTF, FIG. 2B. This bandwidth is more than sufficient for discriminating spectroscopic differences between HbO₂, Hb, water and lipids all of which are greater than 30 nm.¹⁰

Spatial Resolution. The spatial resolution characteristics of the imaging system were determined by a contrast transfer function analysis, a method expressing the ability of an optical system to distinguish between evenly spaced rectangular bars of a resolution target.^{20,21} The Percent Contrast, C, was experimentally determined from the equation:

$$C = ( \frac{{I}_{max} - {I}_{min}}{{I}_{max} + {I}_{min}} ) \times 100 \qquad ( 3 )$$

where Iₘₐₓ is the maximum intensity reflected by a single white bar of the resolution target and Iₘᵢₙ is the minimum intensity from the nonreflecting, dark bar area between the white lines of the resolution target as described in previous work.^{4,7,8}

Many factors contribute to the overall spatial resolution of an optical system, including focal distance, f-stop, depth of field, optical elements, detector pixel dimensions and degree of pixel binning. A standard 1951 USAF resolution target was placed 5.1 cm from the front end of the 10 mm diameter laparoscope mounted to the LCTF (tuned to pass a center wavelength of 750 nm) coupled to the FPA by a 50-mm F-mount lens whose aperture was opened to an f-stop of 1.4. These parameters provide the highest sensitivity for conditions used during surgery and resulted in imaging a 7.6 cm diameter field of view. FIG. 2C the spatial resolving power of the system is plotted against percent contrast for a variety of FPA binning parameters. Binning of adjacent pixels on the FPA prior to digitization was found to increase the speed of data collection and detector sensitivity but reduces image resolution. Imaging a calibrated 1951 USAF resolution target containing line pairs spaced at known distances and spatial resolution, x axis, for a determined percent contrast, C, of the imaging system, y axis. The resolution limit is the minimum distance between distinguishable objects in an image, for example, a group of line pairs. Applying Rayleigh's criterion, the spatial resolution of the laparoscopic hyperspectral imaging system is 0.24, 0.25, 0.26, and 0.27 mm when binning 1×1, 2×2, 3×3 and 4×4 respectively. Indicating the image contrast produced by the system is sufficient for discriminating anatomical structures on the order of 0.24 mm.

Image Acquisition and Analysis. A study protocol approved by the institutional animal care committee for hyperspectral imaging of the biliary tree in three anesthetized 60-pound swine during laparoscopic procedures was followed after administering general anesthesia. The abdomen of the swine was insufflated with CO₂ and the laparoscope placed through a 10 mm port. The gallbladder was initially identified by the surgeon based on visual morphology obtained from conventional laparoscope images. Subsequently, spectroscopic image data were acquired by the near-infrared laparoscopic hyperspectral imaging system.

Spectral data analysis utilizes a Principle Component Analysis, PCA, of the hyperspectral image data cube highlighting relative distributions of different molecular components. First the reflectance spectra from intra-operative tissues are quantified for apparent absorbance, A_{xy}(λᵢ),^{11, 12, 22} defined as the logarithm of the ratio between reflected sample radiation, R_{xy}(λᵢ), and the reflected radiation from a certified 99.9% reflectance standard, (SRT-99-120; Labsphere, Sutton, N.H.),²³ R_{xy}(λᵢ)ₒ, measured at center wavelengths λᵢ for the spatial coordinates x and y:⁴

A_{xy}(λᵢ)=log₁₀(R_{xy}(λᵢ)_{O}/R_{xy}(λᵢ)) (4)

The PCA determines a set of mutually orthonormal vectors spanning the data space describing the detected spectra for each pixel position x,y.²⁴ The magnitude of each of these vectors was encoded as a gray-scale value at each pixel position, forming a 2-dimensional image for each of the principal component vectors. Empirically, the analysis indicated up to 5 principle components contained spectral information corresponding to know absorbance peaks, and therefore 5 principal component images were created from the hyperspectral image cube. The PCA is performed using the Matlab software environment.

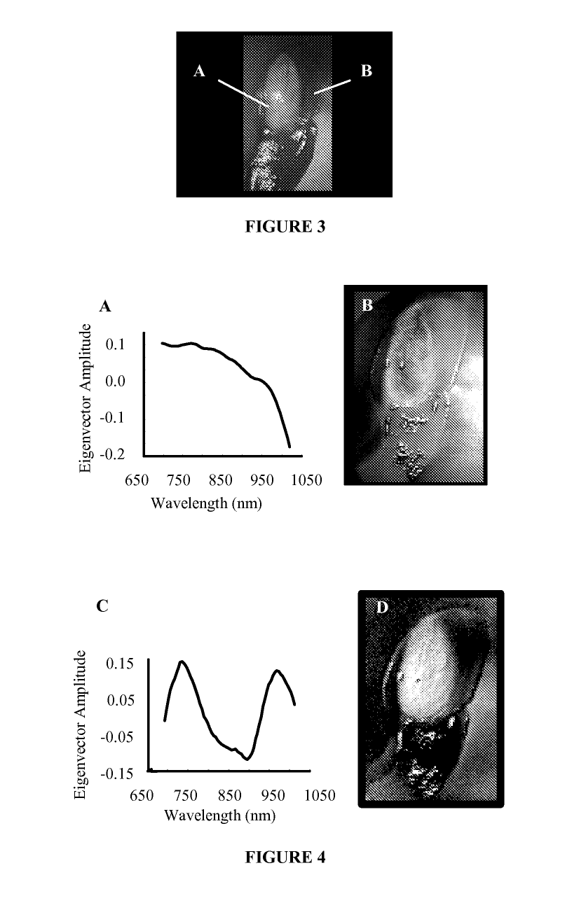

A near-infrared laparoscopic hyperspectral imaging method was developed and characterized for assessing the anatomy and molecular content of tissues during laparoscopic surgery. Images were collected from three anesthetized 60 pound pigs during closed cholecystectomy surgeries. FIG. 3, a white-light picture was taken with a conventional laparoscopic system inserted through a 10 mm port in an anesthetized animal with the purpose of displaying general morphological features and identifying the gallbladder and liver. Subsequently, the near-infrared hyperspectral laparoscopic system was inserted into the same port and spectroscopic images collected. The hyperspectral image cube of the raw data obtained from the intraoperative field of view was analyzed by PCA into its principle component spectra, 5 in this study, and their corresponding images.

Tissue locations within the field of view are visualized by determining a number of principle component images, each of which are gray scale encoded at each pixel by scoring the relative contribution of an inherent principle component to the measured spectrum. Different principal component spectra provide images with different levels of contrast between adjacent tissue structures. For example, the second principle component spectrum, FIG. 4A, differentiates the gallbladder and liver, FIG. 4B; however, the fifth principle component spectrum, FIG. 4C, maximizes contrast and visualizes the gallbladder standing in vivid contrast against the liver, FIG. 4D.

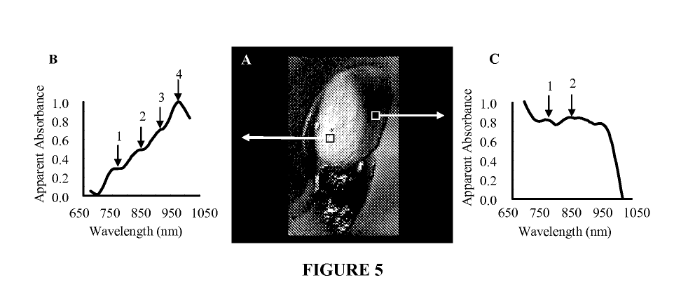

In addition to aiding the surgeon by providing real-time visual display of tissues with enhanced contrast, the hyperspectral imaging technique also provides information about the spatial distribution of tissue chemistry. During laparoscopic cholecystectomy the surgeon can select a region of interest in any principle component image (for example the fifth component, FIG. 5A) and plot the averaged spectrum measured from within that region of the gallbladder (FIG. 5B) versus the spectrum from the surrounding liver, FIG. 5C. It is important to note the original spectra measured at a specific pixel location correspond to the same pixel location in the principle component images. The principle component images enhanced contrast to visualize differing tissue chemistry that is based on and can be confirmed by the measured spectroscopy. Plotting the measured spectra from pixels indicated as being different by the fifth principle component image, FIG. 5, confirms the difference in identity between the gall bladder versus the liver, based on molecular content indicated by the spectra, without the need for any invasive surgical dissection being performed.

Previously reported near-infrared reflectance spectra from biological tissues in vivo show Hb having an absorbance peak at 760 nm, HbO₂ absorbing broadly beyond 800 nm, lipids peaking at 930 nm and water typically peaking at 970 nm.¹⁰ The measured spectrum from the gallbladder, FIG. 5B, shows a peak around 760 nm and a broad band beyond 800 nm, indicating a mixture of Hb and HbO₂. Furthermore, the measured spectrum contains a peak at 930 nm with a peak at 970 nm, indicative of a mixture of lipids and water. The lipid-water mixture is most probably due to the presence of bile contained in the gallbladder, while Hb and HbO₂ was a result of the blood vessels lying over the surface of the gallbladder. In contrast, the measured spectrum from the region of interest in the liver, FIG. 5C, indicates the presence of Hb and HbO₂ with the lipid and water peaks, as expected, are greatly suppressed given the high blood volume content of liver.

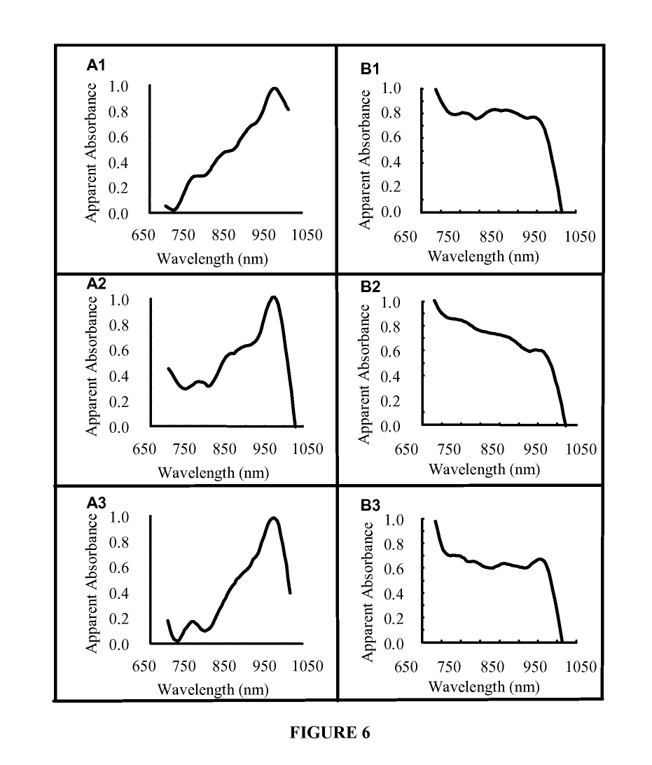

The reflectance spectra from a given tissue type differed slightly from animal to animal due to variations in the angle of illumination and reflected light collection, tissue topography and optical properties. Nevertheless, the salient spectral reflectance features for Hb, HbO₂, lipids and water identified in FIGS. 5B and 5C for the gallbladder and liver, respectively, were similar in all three animals, FIG. 6.

This study illustrates the utility of near-infrared laparoscopic hyperspectral imaging for achieving real-time contrast enhanced visualization of intra-operative tissues without injecting radioactive contrast material. In the surgical setting this technique provides increased contrast of the gallbladder over the standard clinical fiber optic based measurements of local tissue reflectance through a laparoscopic system. Reflectance, point, spectroscopy techniques measuring local average values of the absorption and scattering coefficients of a tissue volume have, in the past, been used for the characterization of tissue composition in a clinical setting by multiple investigators.^{25, 26} A limitation of this approach being the need to scan the tissue area over time to obtain spatial information about the chemical composition of tissues. Spatial scanning methods are time-consuming and result in low resolution images. Integrating recent technological developments in imaging spectrometers, specifically, near-infrared liquid crystal tunable filters and focal plane arrays with surgical laparoscopes overcomes this limitation. That is, by use of an array of detectors one can simultaneously provide rapid image-based spectroscopic assessment of tissue composition from within the body during surgery.

Surgeons train to perform open procedures involving large incisions and identify tissues based on visual morphology and the sense of touch. For example, an artery can be identified by feeling a pulse. Laparoscopic surgery tools have reduced incisions to as little as 10 mm, speeding up patient recovery; however, surgeons have lost tactile feedback creating a need for new tools that will aid them identify intra-operative structures during surgery. The goal of this project was to develop a new imaging platform that will identify anatomical structures based on morphology and spectroscopy during minimally invasive surgical venues without injecting radioactive contrast agents.

A new imaging system consisting of a near-infrared FPA, an LCTF and a laparoscope was constructed and the performance of the imaging system was characterized for minimally invasive surgical venues. The LCTF is tunable over the spectral range of 650 to 1100 nm while the FPA has a spectral response between 200 to 1100 nm, a combination giving a useful imaging system range from 650 to 1090 nm. The wavelength dependent bandwidth of the device was measured as full width at half max and determined from the transmission spectra to be 6.9 nm on average. A 16 bit digitizer produces images with 65,536 shades of gray capable of detecting 0.002% spectral peak amplitude changes. The optimal spatial resolving power ranges between 0.24 to 0.27 mm when binning by 1 and 4, respectively, as determined by the percent contrast method. The FPA detector, with its format of 1340 by 400 detector pixels, is capable of registering 536,000 simultaneously acquired spectra over the sample field of view. PCA methodology was applied to the acquired hyperspectral data yielding images of intraoperative tissues with enhanced contrast, and providing information about molecular composition of the tissue.

These results demonstrate how to make and use the apparatus of the present invention using anesthetized swine undergoing laparoscopic cholecystectomy demonstrated consistent results in three pigs, as well as, the robust utility of the method. The spectroscopic multichannel array detector approach described provides rapid, repetitive visualization of intraoperative tissue, and we expect this method to become a useful tool in aiding surgeons visualize and identify diseased tissues and structures, in vivo, during difficult laparoscopic procedures such as cholecystectomy. The system can be used in a surgical setting for imaging the human gallbladder during cholecystectomy procedures. Use of this laparoscopic hyperspectral imaging apparatus and method provides the skilled artisan with a new class of imaging technology for a variety of minimally invasive surgical procedures.

### EXAMPLE

### Laparoscopic Cholecystectomy

Laparoscopic cholecystectomy is one of the most commonly performed operations in the United States. The most important complication of this operation is injury to the common bile duct, CBD. Injury may occur because dissection in this area is necessarily performed blindly and the CBD may be inadvertently cut. Thus there is a need for a simple method for locating the bile duct system during surgery. Given that there are 500,000 cholecystectomies performed annually; 2,500 common bile duct injuries occur per year, a trend that is increasing. These are serious injuries, resulting in a substantial increase in morbidity and mortality. Patient safety during cholecystectomy would be significantly enhanced if there were imaging devices enabling surgeons to easily see through tissues and visualize various structures prior to, or during, dissection without the need of infusing contrast agents and radiographs. A near infrared laparoscope can be used to enable the operating surgeon to visualize intraportal structures, specifically the common bile duct, while performing the cholecystectomy without contrast injection and radiographs.

Routine intraoperative ultrasonography has been proposed for delineating biliary anatomy. However, the images are not very clear and identifying the relevant anatomy is challenging. Even in relatively small trials of routine intraoperative ultrasonography, major bile duct injuries occurred, demonstrating that the technique falls short as an injury-prevention strategy. Despite being available for many years, intraoperative ultrasound has not been adopted by surgeons for identifying portal anatomy during cholecystectomy. A recent analysis of the National Hospital Discharge Survey (NHDS) and National Inpatient Survey (NIS) reveal that intraoperative lacerations occurring during laparoscopic cholecystectomy have changed little with time. The 5-year average injury rate is 3.1 injuries per 10,000 cholecystectomies. In contrast, as experience with open operations declined, injury rates increased and for the past 5-years have averaged 21.1 injuries per 1,000 open cholecystectomies. Using the NIS, we estimated that an injury adds $5,203 to the initial admission hospital costs. For all bile duct injuries excess health care costs are in excess of $6.5-$13 million per year in the U.S. for the initial hospitalization and far more for the necessary follow-up care. When there is significant inflammation, it is not safe to dissect the bile duct because it cannot be readily visualized. Under these circumstances partial cholecystectomies are performed. This operation is increasing in frequency. When artial cholecystectomy is done, part of the gallbladder is removed and drains are placed. Patients, frequently have multiple hospitalizations, outpatient clinic visits and procedures. Using NHDS and NIS, we determined that the 1,400 partial cholecystectomies performed annually in the U.S. result an extra $7,162 per index admission translating to $10,000,000 in potentially avoidable health care costs. Operating rooms cost about $15/minute to run. Surgeons spend a considerable amount of time dissecting portal structures and trying to identify the biliary system. Substantial health care savings could be achieved (but difficult to estimate) by the development of an imaging system that reduces operating room times and obviates the need to perform intraoperative cholangiograms (at $750 per case).

Development of a biliary imaging system will improve patient safety, has the potential for avoiding the need for partial cholecystectomy and could eliminate bile duct injury as a complication of cholecystectomy. Although relatively infrequent, when bile duct injuries occur they have devastating consequences. No currently available technology substantially reduces the incidence of these complications. There is a pressing need to develop better technologies that would enable surgeons to visualize bile ducts during surgery.

Currently, the only imaging methodology available for intraoperative bile duct visualization is intraoperative cholangiography, IOC, a technique that has changed little over the past 60 to 70 years. The near infrared laparoscopic hyperspectral imaging system of the present invention may be used to visualize different anatomical structures during surgery. The imaging system will be tested for its ability to repeatedly visualize the biliary system in 40 human patients undergoing gallbladder removal. Surgeons using the IR laparoscopic hyperspectral imaging system has greater contrast for identifying intraportal structures while performing cholecystectomy.

The IR (infrared) and NIR (near infrared) laparoscopic hyperspectral imaging tool allows surgeons to “see through” the porta hepatis and visualize the common bile duct during cholecystectomy. The laparoscopic hyperspectral imaging tool can be used to: (1) Examine optical properties of bile ducts, fatty tissues, blood vessels and liver using the IR laparoscopic hyperspectral imaging system and create a database of in vivo, intraoperative human tissue spectra. (2) Evaluate the utility and repeatability of the IR laparoscopic hyperspectral imaging system for visualizing the human biliary system in routine open and laparoscopic surgery. (3) Determine the specificity of the IR hyperspectral imaging system for differentiating bile from fat. (4) Evaluate the fluorescence utility of indocyanine green to increase the contrast between bile ducts and surrounding fat.

The problem faced by surgeons performing gallbladder operations is that important structures are embedded in fat and other tissues and are, consequently, hidden from view. Surgeons rely on technical skills developed after many years of training to dissect these otherwise hidden structures without harming the critical ones. The only imaging aid currently available for intraoperative bile duct visualization is intraoperative cholangiography. Current efforts to minimize the risk of common bile duct injuries, CBDI, involve the use of intraoperative cholangiography (IOC) a technique developed more than 60 years ago. IOC requires identification and cannulation of the cystic duct and obtaining a fluoroscopic image of the biliary tree. Although this provides the surgeon with a map of the biliary system, subsequent dissection remains blind and only improved in that the surgeon has knowledge of the approximate location of important anatomical landmarks. Application of IOC to every procedure has been shown to only modestly reduce the rate of bile duct injury. Routine application of IOC is not cost effective from the perspective of a risk reduction strategy to minimize the risk of bile duct injury. Ultrasound techniques have not proved effective for intraoperative bile duct imaging.

Hyperspectral imaging techniques can be used to identify the biliary structures during gallbladder surgery. We have demonstrated that blood, bile and fat all have unique infrared (IR) spectra enabling us to visualize and identify portal structures. We have developed an infrared imaging system that enables us to see deeply into the porta to visualize and identify the bile duct. This system is similar to conventional video imaging and has been adapted to laparoscopic surgery equipment. This system will enable surgeons to visualize the biliary anatomy prior to initiating dissection and has the potential for completely eliminating the risk for bile duct injury and potentially reducing the incidence of partial cholecystectomy procedures performed because the bile ducts could not be imaged when a cholecystectomy was attempted. Because early identification of the biliary anatomy will greatly speed the performance of these operations and injuries will be minimized patient safety will be enhanced and health care costs will be reduced.

The focus of this example is to use the innovative infrared, IR hyperspectral imaging system, during surgery, for differentiating the biliary tree from other anatomical structures obviating the need for dissection of the cystic duct, contrast injection and radiographs. This digital spectroscopic imaging system will allow for identification of the portal biliary tree using a novel focal plane array and liquid crystal tunable filter technology attached to conventional laparoscopic surgical instrumentation.

The goal of this research plan is to enable the operating surgeon to visualize intraportal structures, specifically the common bile duct, while performing the cholecystectomy without contrast injection and radiographs. Currently, excluding the use of contrast injection and radiographs no technology exists for visualizing the bile duct. A newly developed infrared laparoscopic hyperspectral imaging system, (a device existing in the laboratory of the PI) will be utilized for visualizing different anatomical structures during surgery. The varying amounts of chromophores inherently present in vivo tissue and sensitive to near infrared radiation will be detected using a new digital near infrared (NIR) focal plane array (FPA) detector and liquid crystal tunable filter (LCTF) coupled to conventional laparoscopic surgical technology for exposing anatomical structures normally hidden from the surgeon's view. Conventional surgical laparoscopic technology will be used as a means for bringing images of intraportal structures during surgery to the attached hyperspectral imaging system that will detect and collect a spectrum at each pixel detector of the focal plane array. Illuminating tissue with near infrared light reflects spectral characteristic of oxyhemoglobin (HbO₂), deoxyhemoglobin (Hb), lipids and water (H₂O). Applying chemometric methods results in images encoded either inherent principle components or for varying quantities of known reference spectra. Since differing anatomical structures inherently contain varying amounts of molecules know to exhibit spectral absorption peaks when illuminated with near infrared light, applying chemometric methods can produce an image visualizing the location of these molecules and their associated anatomical structures.

The goal of the prospective research is to evaluate the innovative medical imaging technology, IR laparoscopic hyperspectral imaging, intra-operatively during cholecystectomy. It is hypothesized that the IR laparoscopic hyperspectral imaging system will enable surgeons to “see through” the porta hepatis and visualize the common bile duct during cholecystectomy without infusing contrast agents or radiographs. The imaging system will be tested for its ability to repeatedly visualize the biliary system in 40 patients undergoing gallbladder removal. It is expected that surgeons using the IR laparoscopic hyperspectral imaging system will find this to be a useful tool for identifying intraportal structures while performing the cholecystectomy.

Study Population. The study population will consist of both animal and human patients. The animal population will consist of 6 60-80 pound swine. Patients will be selected from the Veterans Affairs Hospital, Dallas where cholecystectomies are performed in excess of 1800 annually on a mostly indigent population. Hispanic comprises 40% of patients, African-Americans 40% and Caucasians 20%. For those undergoing cholecystectomy, 75% are female. Children are cared for at separate children's facility and are not anticipated to enroll into this study. There will be no other exclusions in terms of patients enrolled.

Study Protocol. As indicated by the study specific aims and FIG. 9 the protocol will perform both animal and human studies with the majority of emphasis being placed on human studies. Initially 6 swine will be imaged, approximately one per week followed by an evaluation period and preparation for human studies. Any patient undergoing a gallbladder removal surgery at the Veterans Affairs Hospital, Dallas will be eligible to participate in the study. To evaluate the surgical robustness of this new class of imaging technology the study plans on imaging, intraoperatively, a total of 40 patients during surgery. The initial study period will image 20 patients over a 10 week period, followed by a second 10 week period imaging the remaining 20 patients. These two human imaging periods may be separated by a mid-study evaluation consisting of data analysis and a review of the data collection techniques. The final month will consist of final evaluation and submitting the results to conferences and high impacting clinical journals.

Procedures for the Human Study. The ultimate goal of this study is to enable the surgeon to “see through” the porta hepatic and visualize the common bile duct during cholecystectomy. A new imaging platform, laparoscopic hyperspectral imaging, will be translated to surgery in the VA Hospital at Dallas where human patients will be imaged intra-operatively using the IR laparoscopic hyperspectral imaging system. The spectroscopic image data collected by the hyperspectral system from live anesthetized human patients will be used for examining optical properties of human intraportal structures. Specifically, bile ducts, fatty tissues, blood vessels, gallbladder, stomach and liver tissue. The resulting database of in vivo tissue spectra will be applied toward identifying the biliary system within the porta based on the spectra collected during surgery. To evaluate the utility of the IR laparoscopic hyperspectral imaging system for visualizing the human biliary system during routine open and laparoscopic surgery. A principle component analysis, PCA, will be applied to the hyperspectral image data to determine the contribution of inherent principle component spectra for the measured spectrum at each pixel that is scaled to produce a gray scale image. The PCA image is expected to have greater contrast than a standard digital picture for identifying surgical landmarks such as the stomach, gallbladder and liver with the ability to visualize the bile ducts within the porta. Spectra and principle component images from all 40 patients will be evaluated for consistency to determine the repeatability of the measure.

Any patient undergoing a gallbladder removal surgery at the VA Hospital at Dallas will be eligible to participate in the study. Standard routine surgical procedures for cholecystectomy will be followed with the addition of collecting digital laparoscopic photos and IR hyperspectral imagery with the purpose of visualizing the biliary tree during the surgical procedure. Images will be collected from a total of 40 human patients. Initially 20 patients undergoing open cholecystectomy will be studied. An incision on the abdomen is made by the surgeon, open laparoscopy. The liver will be retracted and intraportal structures such as the liver, gallbladder, cystic duct, common bile duct and common hepatic duct along with any other tissue such as fat and blood vessels will be visually identified by the surgeon. Pictures of the identified structures will be pictured using a standard digital camera, Cannon Power Shot S3IS. Subsequently the IR laparoscopic hyperspectral imager will be positioned and hyperspectral image data collected. A similar procedure will be used in the final 20 patients except that the abdomen will not be opened, instead, the procedure will be done laparoscopically. Digital pictures will be provide by the standard surgical laparoscope, a Karl Storz system, that is being used for the procedure and the hyperspectral images will be provide by the IR hyperspectral imaging system with a laparoscope attached for imaging the porta from within the body. It is expected that the images collected by the IR laparoscopic hyperspectral imaging system will clearly visualize the biliary tree, which can not be seen when compared to a standard digital picture. The contrast (as determined in previous work,²⁷) for the bile ducts produced by the standard laparoscoic system versus those of the hyperspectral imager will be compared.

To further elucidate the specificity of the IR hyperspectral imaging system to differentiate bile from fat, samples of human bile and fat will be collected from each patient and examined in the laboratory. Firstly, samples of bile will be loaded into a capillary tube and immersed into an intralipid solution, mimicking the optical properties of fat, and imaged with the hyperspectral imaging system to determine the maximum penetration depth of the system. The capillary will be imaged at the intralipid surface (zero depth) and then lowered and imaged below the intralipid solution surface at 0.1 mm increments until the bile spectral peaks are twenty percent of those measured at the surface, defining the penetration depth of the hyperspectral system.

Secondly, samples of bile and fat will be loaded into separate cuvettes that are placed into a spectrophotometer for determining the absorbance and scattering coefficients as a function of wavelength as described previously by Zonios.³³⁻³⁴ The coefficients are then used as input parameters for a Monte Carlo algorithm which will simulate the bile-fat tissue and light illumination geometries of the physical experiments. These simulations will be used to validate the experimentally measured sensitivity and specificity of the hyperspectral imaging system for differentiating bile from fat intraoperatively. Dr. Hanli Liu has offered us time for using the near infrared spectrophotometer for determining absorbance and scattering coefficients. The intralipid solution and equipment for lowering the capillary tube of bile at various depths below the intralipid surface will also be provided by Dr. Hanli Liu. Monte Carlo simulations to model tissue and illumination geometry will be performed by Dr. George Alexandrakis.

Procedures for the Swine Study. When the biliary structures are inflamed, edematous tissue may obscure the specificity of the hyperspectral imager to visualize the bile ducts. For these difficult cases we propose infusing indocyanine green (ICG) into the hepatic artery. ICG is a fluorochrome that emits light, fluoresces, with a bandpass of 780-820 nm when excited by a bandpass of 716-756 nm.³⁵ After intravenous injection ICG is rapidly taken up by the liver and then excreted unchanged into the bile. Augmenting the hyperspectral imager by adding a short pass optical filter into the beam path at the source and illuminating only with wavelengths of light below 756 nm will excite ICG in the bile ducts and decrease cross-talk between excitation and emission channels. Collecting a series of images between 780 to 820 nm with the hyperspectral imaging system, we hypothesize images collected after ICG infusion will fluoresce within the bile ducts visualizing their location.

Three swine will be used in open procedures and three swine will be used in laparoscopic operations. The swine will be anesthetized and securely positioned on a standard surgical table in which the abdomen is exposed. In the first three swine an incision on the abdomen is made by the surgeon, open laparoscopy. The liver will be retracted and the porta will be imaged with the IR laparoscopic hyperspectral imaging system. Secondly, a short pass filter is added into the beam path of the source thus augmenting the IR laparoscopic hyperspectral imager. A bolus of ICG is injected intravenously (as is currently practiced for assessing cardiac output and liver function³⁶ and the augmented hyperspectral imaging system will start monitoring the porta for fluorescence indicating the location of the bile ducts. A similar procedure will be used in the final three swine except that the abdomen will not be opened, instead, the procedure will be done laparoscopically. It is expected that the images collected by the augmented IR laparoscopic hyperspectral imaging system and ICG will have an increased contrast for the bile ducts when compared to the standard IR laparoscopic hyperspectral imaging system.

The innovative IR laparoscopic hyperspectral imaging tool is a simpler method enable surgeons to “see through” the porta hepatis and visualize the common bile duct during cholecystectomy obviating the need for radiographs. Quantitative measures from hyperspectral image data will be determined using chemometric methods, specifically, principle component analysis (PCA) and a multivariate least-squares deconvolution of the hyperspectral data cube; sampling detector pixels to define rectangular area, as described in greater detail in previous publications,^{20,26} within the region of interest in this case the biliary tree and surrounding intraportal structures. The data will be analyzed using desktop computers running Matlab and Microsoft Excel. The logistics and budgeted clinical staff for this study limit the maximum number of subjects to 40; however, based on data accumulated from previously published hyperspectral imaging studies,^{26-27,37} that reveal measurements made by averaging 1000-odd pixel values have very low variability. A power calculation for within subject comparisons indicates 40 subjects are sufficient for detecting very small changes in mean values. A student-t test will be utilized to determine those parameters that are significantly altered between biliary tree and other intraportal structures such as the liver, gallbladder, and other tissue for example blood vessels and fat. Cross-correlations will be studied to determine complementary, parallel, or reciprocal relationship, especially a structural, functional, or qualitative correspondence between regular digital pictures and the hyperspectral images. ANOVA (Analysis of Variance) will be applied to conduct comparisons as an indication of the reliability of the measure over time. A general linear mixed model for repeated measures will be used to evaluate hyperspectral imaging data collected over time for each patient. This method is particularly useful in assessing within-subject change collected by hyperspectral imaging while accounting for factors and treatment effects varying across multiple subjects.³⁸ All comparisons will be performed with Type 1 error set to a probability of 0.05. Collected digital data and images will be coded and stored on a hard drive of a secure server located in the laboratory of the PI for subsequent analysis. The server performs data backups automatically and is routinely checked.

Infrared Laparoscopic Hyperspectral Imaging of Bile Ducts. Hyperspectral images were collected with the infrared hyperspectral imaging system (FIG. 1) of the gallbladder, cystic duct and liver in six 60-80 pound swine and 1 human. Tissue locations within the field of view are visualized by determining a number of principle component images, each of which are gray scale encoded at each pixel by scoring the relative contribution of an inherent principle component to the measured spectrum. In addition to aiding the surgeon by providing real-time visual display of tissues with enhanced contrast, the hyperspectral imaging technique also provides information about the spatial distribution of tissue chemistry. During laparoscopic cholecystectomy the surgeon can select a region of interest in any principle component image and plot the averaged spectrum measured from within that region, for example, the gallbladder versus the spectrum from the surrounding liver. It is important to note the original spectra measured at a specific pixel location correspond to the same pixel location in the principle component images. The principle component images enhanced contrast to visualize differing tissue chemistry that is based on and can be confirmed by the measured spectroscopy.

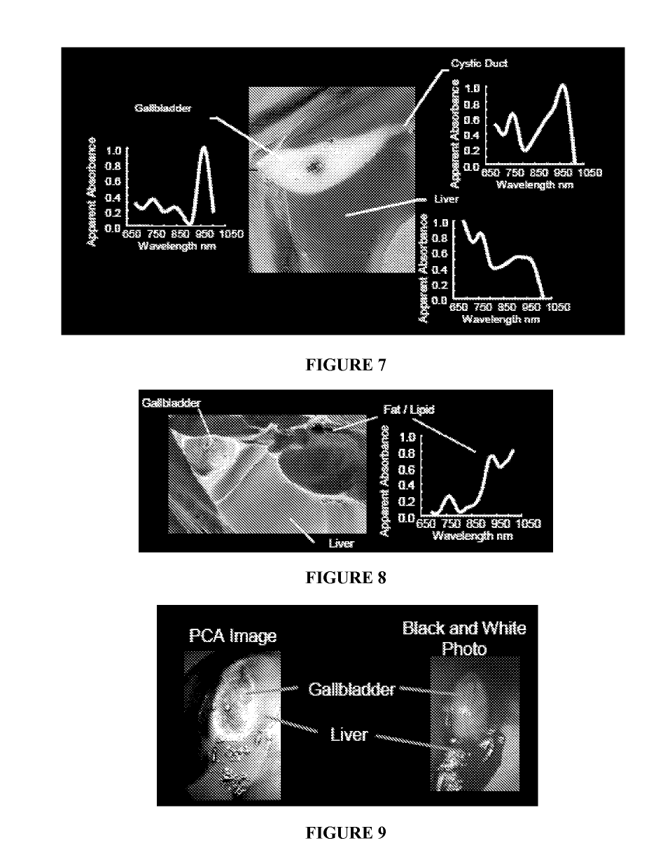

FIG. 7. A PCA image visualizing the Gallbladder, liver, and cystic duct and their associated spectra. The liver was retracted, spectroscopic image data was collected and analyzed using principle component analysis (PCA) indicating the presence of a variety of differing spectra. Referring to known spectra for oxyhemoglobin, water and lipids, the intraportal structures can be identified based on measured spectral peaks indicating a molecular presence within the tissue that is not pictured using a standard camera. The gallbladder in this image contains bright pixels representing a spectrum containing a strong absorption peaks for water at 970 nm, and smaller absorptions for deoxyhemoglobin at 760 nm and the broad oxyhemoglobin beyond 800 nm. The gray cystic duct contains a convoluted spectrum containing oxy-, deoxyhemoglobin, water, and a lipid shoulder at 930 nm. Oxy- and deoxyhemoglobin is mostly absorbed by blood in the microvasculature on the surface of the structure, while the strong water and lipid absorbance is most likely due to the bile containing these two molecules within the collecting duct. The darker imaged liver contains spectra containing characteristic peaks for oxy- and deoxyhemoglobin. Since this swine had been euthanized 5 to 10 minutes prior to the imaging the images were expected to contain a significant amount of deoxyhemoglobin as indicated by the spectrum.

FIG. 8. Fat and its associated spectrum lying along the stomach within a PCA image. Imaging from a different perspective the gallbladder, liver and fatty tissue around the stomach is visualized. The fat is mapped within the PCA image as darker pixels that are associated with spectra absorbing strongly at the characteristic

FIG. 9. A PCA image from data collected with the infrared hyperspectral imager is compared to a conventional black and white photo of the gallbladder taken with a standard surgical Karl Storz laparoscopic system. Both images were collected from the same live anesthetized pig while performing a closed cholecystectomy. The PCA image provides better contrast for the gallbladder (without infusing any contrast agent) and the associated hyperspectral data provides spectra indicating the presence of molecular components contained in bile from pixels imaging the gallbladder. Spectra from PCA pixels viewing the gallbladder contained absorbance peaks for oxy-, deoxyhemoglobin and water while liver spectra contained oxy- and deoxyhemoglobin absorbance peaks with water being significantly suppressed.

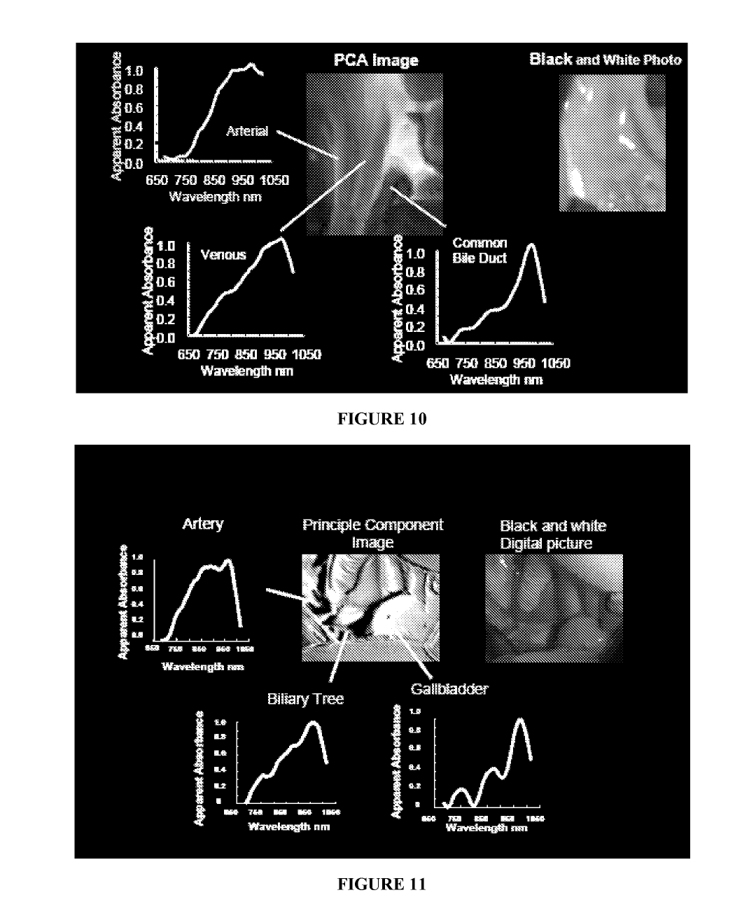

FIG. 10, the PCA image visualizing portal structures prior to dissection, left image, and a standard black and white photo, of the same porta, taken with a standard surgical laparoscopic system, right image, during a laparoscopic procedure in live anesthetized pigs. The artery, vein and common bile duct were identified through connective tissue by measuring near-infrared spectra. An artery indicated by spectra with a very pronounced broad oxyhemoglobin peak around 800 nm and a water peak at 970 nm. A venous structure produces spectra with a deoxyhemoglobin shoulder at 760 nm and a broad oxyhemoglobin peak around 800 nm and a water peak at 970 nm. The common bile duct is identified by spectra containing a lipid shoulder at 930 nm and a prominent water peak at 970 nm. The standard black and white photo imaging the same porta shows the connective tissue; however, individual structures are obscured from view. After imaging was complete the connecting tissue was surgically dissected which confirmed the location of an artery on the far left side, a venous structure in the middle and the common bile duct entangled to the right side.

The PCA image in FIG. 11 visualizes the first human bile duct through an undissected porta, and without using a contrast agent. In addition other land marks such as the gallbladder and an artery are identified. The PCA image is found to have greater contrast for visualizing the bile duct than a standard black and white picture. Similar to the animal studies the spectra measured from the human mapped the location of the artery, biliary tree and gallbladder. As seen in FIG. 11, the arterial spectrum shows the characteristic oxyhemoglobin absorption between 800 to 900 nm and water peak at 970 nm. Absence of deoxy-hemoglobin peak at 760 nm is an indication that the artery has little or no deoxyhemoglobin component. The measured spectrum from the biliary tree shows a spectral shoulder around 930 nm indicating the presence of lipids and a water peak at 970 nm. The lipid mixture is most likely due the presence of bile in the biliary structures. The measured spectrum from the gallbladder, shows a peak around 760 nm and a broad band beyond 800 nm, indicating a mixture of Hb and HbO₂; most likely due to surface blood vessels. Furthermore, the measured spectrum contains a peak at 970 nm, indicative of water.

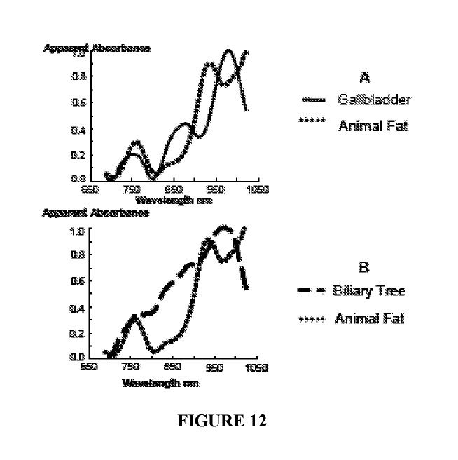

Examining the principle component spectra sampled from fat and the gallbladder, FIG. 12A, shows the gallbladder having a predominant water peak, 970 nm, and little to no absorption due to fat, 930 nm. Comparing a spectrum from the biliary tree, FIG. 12B, shows a characteristic peak for water at 970 nm that has been broadened to 930 nm most likely due to a presence of lipids. In contrast, fat has weak water absorption while having a significant lipid peak at 930 nm. This massive 50 nm spectral difference between water and fat gives the hyperspectral imaging system the specificity of imaging bile ducts within fatty tissue; bile contains a significant water peak versus fat.

We demonstrate using the IR hyperspectral imaging system with commercially available laparoscopic surgery instruments. We found that biliary structures have unique IR spectra that can be used for their identification. There are indications that bile and fat can be spectrally differentiated. To further elucidate the specificity of the system to differentiate these components further study of light-tissue interactions are planned. In cases where the fatty tissue scatters the IR radiation making it difficult to measure spectra and hence identifying the underlying structures, we will examine the feasibility of a contrast approach—i.e. an intravenous injection of a substance, indocyanine green that collects in bile and can be imaged by our IR hyperspectral system. In the course of obtaining these preliminary images the system requires several seconds to acquire the spectroscopic image data; movement artifact can occur in this time. Thus, during surgery, the porta may require stabilization and we are developing sub-registration software for detecting and correcting pixel drifts. The preliminary data was collected from swine and 1 human patient. Future studies are needed for imaging the cystic and hepatic ducts to test reliability of the measure.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

(1) Livingston, E. H. American Journal of Surgery 2004, 188, 105-110.
(2) Livingston, E. H.; Rege, R. V. Journal of the American College of Surgeons 2005, 201, 426-433.
(3) Mirrizzi Surg Gynaecol Obstet 1937, 65, 702-710.
(4) Zuzak, K. J.; Schaeberle, M. D.; Lewis, E. N.; Levin, I. W. Analytical Chemistry 2002, 74, 2021-2028.
(5) Cheong, W. F.; Prahl, S. A.; Welch, A. J. Ieee Journal of Quantum Electronics 1990, 26, 2166-2185.
(6) Hebden, J. C.; Arridge, S. R.; Delpy, D. T. Physics in Medicine and Biology 1997, 42, 825-840.
(7) Zuzak, K. J.; Gladwin, M. T.; Cannon, R. O.; Levin, I. W. American Journal of Physiology-Heart and Circulatory Physiology 2003, 285, H1183-H1189.
(8) Zuzak, K. J.; Schaeberle, M. D.; Gladwin, M. T.; Cannon, R. O., 3rd; Levin, I. W. Circulation 2001, 104, 2905-2910.
(9) Zuzak, K. J.; Schaeberle, M. D.; Levin, I. W.; Lewis, N. E.; Freeman, J.; McNeil, J. D.; Cancio, L. C. IEEE 1999, 2, 1118.
(10) Taroni, P.; Pifferi, A.; Torricelli, A.; Cubeddu, R. Opto-Electronics Review 2004, 12, 249-253.
(11) Hajizadehsaffar, M.; Feather, J. W.; Dawson, J. B. Physics in Medicine and Biology 1990, 35, 1301-1315.
(12) Knoefel, W. T.; Kollias, N.; Rattner, D. W.; Nishioka, N. S.; Warshaw, A. L. Journal of Applied Physiology 1996, 80, 116-123.
(13) Lewis, E. N.; Gorbach, A. M.; Marcott, C.; Levin, I. W. Applied Spectroscopy 1996, 50, 263-269.
(14) Lewis, E. N.; Levin, I. W. Applied Spectroscopy 1995, 49, 672-678.
(15) Morris, H. R.; Hoyt, C. C.; Treado, P. J. Applied Spectroscopy 1994, 48, 857-866.
(16) Schaeberle, M. D.; Morris, H. R.; Turner, J. F.; Treado, P. J. Analytical Chemistry 1999, 71, 175a-181a.
(17) Sowa, M. G.; Payette, J. R.; Mantsch, H. H. Journal of Surgical Research 1999, 86, 62-69.
(18) Kidder, L. H.; Kalasinsky, V. F.; Luke, J. L.; Levin, I. W.; Lewis, E. N. Nature Medicine 1997, 3, 235-237.
(19) Lewis, E. N.; Treado, P. J.; Reeder, R. C.; Story, G. M.; Dowrey, A. E.; Marcott, C.; Levin, I. W. Analytical Chemistry 1995, 67, 3377-3381.
(20) Inoue, S.; Spring, K. R., Eds. Video Microscopy: The Fundamentals, Second ed.; Plenum Press New York, 1997.
(21) Kline, N.J.; Treado, P. J. Journal of Raman Spectroscopy 1997, 28, 119-124.
(22) Koetuim, G. Reflectance Spectroscopy, Principles, Methods, Application; Springer Verlagt New York, 1969.
(23) Springsteen, A. Spectroscopy 2000, 15, 20-27.
(24) Geladi, P.; Grahn, H. Multivariate Image Analysis; John Wiley & Sons Ltd, 1996.
(25) Doornbos, R. M. P.; Lang, R.; Aalders, M. C.; Cross, F. W.; Sterenborg, H. J. C. M. Physics in Medicine and Biology 1999, 44, 967-981.
(26) Pham, T. H.; Bevilacqua, F.; Spott, T.; Dam, J. S.; Tromberg, B. J.; Andersson-Engels, S. Applied Optics 2000, 39, 6487-6497.

What is claimed is:

1. A hyperspectral surgical laparoscope comprising:
an illuminated laparoscope;
a liquid crystal tunable filter generally center-mounted on the laparoscope and positioned to collect back-reflected light from a target;
a relay lens generally center-mounted on the laparoscope to focus light from the liquid crystal tunable filter; and
a focal plane array generally center-mounted on the laparoscope, wherein light that is reflected from the target is imaged on the focal plane array and captured as a digital data cube.

2. The laparoscope of claim 1, wherein the illuminated laparoscope delivers continuously tunable light in the near-infrared spectral range of 650-1100 nm.

3. The laparoscope of claim 1, wherein light at the focal plane array has a mean bandwidth of 695 nm.

4. The laparoscope of claim 1, wherein the focal plane array is a high-sensitivity, back-illuminated, deep depleted charged coupled device.

5. The laparoscope of claim 1, wherein the digital data cube is formatted into a three dimensional hyperspectral image cube and processed using principle component analysis.

6. The laparoscope of claim 1, wherein the digital data cube is processed to enhance the contrast between chemically different anatomical structures due to chromophores inherent to the target, chromophores that have been added to the target or both.

7. The laparoscope of claim 1, wherein the target comprises an intraperitoneal tissue, a gall bladder or a bile duct.

8. The laparoscope of claim 1, wherein the laparoscope is connected to a light source comprising a visible to near infrared liquid light guide.

9. The laparoscope of claim 1, wherein the laparoscope is connected to a light source comprising a 250-W quartz-tungsten-halogen broadband source.

10. The laparoscope of claim 1, further comprising an ultraviolet radiation filter positioned between a light source and the target.

11. The laparoscope of claim 1, wherein the liquid crystal tunable filter comprises an electronically controlled and continuously tunable filter with 150 ms tuning response time and a clear 20 mm aperture.

12. A near-infrared hyperspectral surgical laparoscope comprising:
an near-infrared illuminated laparoscope;
a liquid crystal tunable filter generally center-mounted on the laparoscope and positioned to collect back-reflected light from a target;
a relay lens generally center-mounted on the laparoscope to focus light from the liquid crystal tunable filter; and
a focal plane array comprising a high-sensitivity, back-illuminated, deep depleted charged coupled device generally center-mounted on the laparoscope, wherein light that is reflected from the target is imaged on the focal plane array and captured as a digital data cube.

13. The laparoscope of claim 12, wherein the laparoscope delivers continuously tunable light in the near-infrared spectral range of 650-1100 nm.

14. The laparoscope of claim 12, wherein light at the focal plane array has a mean bandwidth of 695 nm.

15. The laparoscope of claim 12, wherein the digital data cube is formatted into a three dimensional hyperspectral image cube and processed using principle component analysis.

16. The laparoscope of claim 12, wherein the digital data cube is processed to enhance the contrast between chemically different anatomical structures due to chromophores inherent to the target or chromophores that have been added to the target.

17. An imaging method to identify a biliary tree structure comprising:
imaging the biliary tree structure with a laparoscope comprising:
a liquid crystal tunable filter (LCTF) positioned to collect back-reflected light from a target;
a relay lens center-mounted on the laparoscope to focus light from the liquid crystal tunable filter; and
a focal plane array (FPA), wherein light that is reflected from the target is imaged on the focal plane array and captured as a digital data cube; and
processing the digital data cube to enhance the contrast between chemically different anatomical structures due to chromophores at the target.

18. The method of claim 17, wherein the illuminated laparoscope delivers continuously tunable light in the near-infrared spectral range of 650-1100 nm.

19. The method of claim 17, wherein light at the focal plane array has a mean bandwidth of 6.95 nm.

20. The method of claim 17, wherein the focal plane array is a high-sensitivity, back-illuminated, deep depleted charged coupled device.

21. The method of claim 17, wherein the digital data cube comprises a three dimensional hyperspectral image cube.

22. The method of claim 17, wherein the digital data cube is formatted into a three dimensional hyperspectral image cube and processed using principle component analysis.

23. The method of claim 17, wherein the digital data cube is processed to enhance the contrast between chemically different anatomical structures due to chromophores inherent to the target.

24. The method of claim 17, wherein the digital data cube is processed to enhance the contrast between chemically different anatomical structures due to chromophores that have been added to the target.

25. The method of claim 17, wherein the target comprises an intraperitoneal tissue, a gall bladder or a bile duct.

26. The method of claim 17, wherein the laparoscope is connected to a light source comprising a visible to near infrared liquid light guide.

27. The method of claim 17, wherein the laparoscope is connected to a light source comprising a 250-W quartz-tungsten-halogen broadband source.

28. The method of claim 17, further comprising an ultraviolet radiation filter positioned between the light source and the target.

29. The method of claim 17, wherein the liquid crystal tunable filter comprises an electronically controlled and continuously tunable filter with 150 ms tuning response time and a clear 20 mm aperture.

30. The method of claim 17, wherein the laparoscopy is open.

31. A system for laparoscopic cholecystectomy comprising:
imaging laparoscopic surgery imaging the intraportal structure with a near infrared laparoscope that comprises:
a liquid crystal tunable filter (LCTF) positioned to collect back-reflected light from a target;
a relay lens center-mounted on the laparoscope to focus light from the liquid crystal tunable filter; and
a focal plane array (FPA), wherein light that is reflected from the target is imaged on the focal plane array and captured as a digital data cube;
processing the digital data cube to enhance the contrast between chemically different anatomical structures due to chromophores at the target; and
removing those tissue that are identified in need of removal.

32. The method of claim 31, wherein the laparoscopy is open.
